(19) United States
(12) Patent Application Publication
(10) Pub. No.: US 2024/0050400 A1
(43) Pub. Date: Feb. 15, 2024

(54) Use of the Sesquiterpene Lactone Tomentosin in the Treatment of Tumors Caused by Lymphoid Cell Lines

(71) Applicant: Universita Degli Studi Di Sassari, Sassari (IT)

(72) Inventors: Luigi Podda, Sassari (IT); Claudio Fozza, Sassari (IT); Patrizia Virdis, Sassari (IT); Maria Rosaria De Miglio, Sassari (IT); Giorgio Antonio Mario, Sassari (IT); Luigi Marco Bagella, Sassari (IT); Rossana Migheli, Sassari (IT)

(21) Appl. No.: 18/552,432

(22) PCT Filed: Mar. 16, 2022
(86) PCT No.: PCT/IB22/52375

§ 371 Date: Sep. 25, 2023

(30) Foreign Application Priority Data
Mar. 25, 2021 (IT) 102021000007220

(51) Int. Cl. A61K 31/365; A61P 35/00

(52) U.S. Cl. CPC A61K 31/365; A61P 35/00

(57) ABSTRACT

The present invention relates to the use of sesquiterpene lactones isolated from extracts of Inula viscosa in the treatment of tumors; in particular, the use of the sesquiterpene lactone Tomentosin in the treatment of lymphoproliferative tumoral diseases, such as, for example, myelomas and/or lymphomas, caused by lymphoid cell lines.

### TECHNICAL FIELD

The present invention relates to the use of sesquiterpene lactones isolated from extracts of Inula viscosa in the treatment of tumors; in particular, the use of the sesquiterpene lactone Tomentosin in the treatment of lymphoproliferative tumoral diseases, such as hematic tumors, e.g. myelomas and/or lymphomas, caused by lymphoid cell lines.

### BACKGROUND ART

While they have led to survival improvement over the years, therapies for oncohematic diseases are continuously under development to respond to many needs that still have to be fulfilled. In fact, several diseases (acute and chronic leukemias, lymphomas, myelomas) still show a high mortality rate notwithstanding the advances made in the research and development of new drugs.

New therapies are therefore needed which can lead to recovery while showing low body toxicity. In other words, it is still necessary to develop medicines, other than traditional (and very toxic) chemotherapy drugs, which can selectively act upon the molecular mechanisms of the disease while preserving the other organs.

In particular, acute and chronic lymphoproliferative diseases are diseases that affect all age groups, even though some forms thereof mainly affect specific age groups (for example, aggressive lymphomas are more frequently observed in adolescents and young adults, while multiple myeloma is more often found in older age groups).

Some of these diseases are curable, while others are considered as incurable, although they can be treated to a certain extent; this means that the available therapies lead to disease remission and/or chronicization, while however still showing a high rate of relapse and mortality. Numerous advances in terms of biological knowledge have been made since the early 2000's, which have led to increasingly “targeted” therapies acting upon sick cells while “sparing” healthy ones. In the course of about 20 years, survival to these diseases has largely improved, but some of them are still incurable, and much research is currently going on in an attempt to find new molecules that can, whether alone or in association, further improve the response and survival rates.

As far as the drugs currently employed are concerned, we have moved from using classic chemotherapy drugs to using increasingly targeted drugs acting upon the immune system and also upon the medullary microenvironment, i.e. acting in such a way as to improve the immune response of the body to the tumor.

In addition to improving the quality of the treatments, these achievements have also resulted in an increased number of patients who can take advantage from them, in that they have, de facto, broadened the age range for which these drugs can be used.

While the cellular metabolic pathways are widely known, the knowledge of the metabolic pathways of neoplastic lymphoid cells is still lacking, and therefore, unfortunately, almost all drugs currently in use also affect normal lymphoid cells, which play a fundamental role in the response to infections.

For this reason, the goal is to find molecules which show low toxicity on normal cells while being more specifically active upon tumor cells.

This is why research has focused on metabolic pathways and the interaction between lymphoid cells and the microenvironment, which normally, and also in neoplastic diseases, co-operate by exchanging “messages” that promote the development, progress, drug resistance and relapse of the disease.

Many molecules exist which mainly act upon sick cells or the microenvironment but very often, after a positive initial response, reach a phase of “refractoriness” to therapies, which is usually connected to mutations and/or development of new neoplastic clones, which become resistant to the drugs that have been used until then. This fact has led to continuous search for and/or development of new molecules, or variations of a “founder” molecule with subsequent modifications concerning affinity, conformation, etc., in order to try to re-establish and/or increase and/or improve a decreased or lost response (the so-called 1st, 2nd, 3rd, . . . generation molecules).

### Shortcomings of the Prior Art and Technical Problem

As described above, the need is still felt for finding (new or modified) molecules which make it possible to, whether due to a different action mechanism or a different metabolic engagement conformation, overcome the problem of refractoriness to therapies; particularly against tumors caused by cells of the lymphoid lineage, which are significantly resistant to current therapies.

It is the object of the present invention to provide an adequate solution to the above-highlighted technical problem.

The Tomentosin and Inuvisculide compounds are sesquiterpene lactones isolated from I. viscosa (L.) Aiton, which has long been used in popular medicine due to its anti-inflammatory, anti-helmintic, anti-pyretic, anti-septic and anti-phlogistic properties, as well as in the treatment of diabetes and pulmonary disorders. Recently, several studies have shown potential anti-cancer effects of sesquiterpene lactones, demonstrating how Tomentosin and Inuvisculide exert anti-cancer effects on some types of human cancer cell lines. For example, Rozenblat et al. have shown a strong pro-apoptotic effect of both sesquiterpenes on aggressive human melanoma cell lines. Furthermore, Tomentosin exerts an anti-cancer effect, mainly by inducing death by apoptosis, in gastric cancer, cervical cancer and osteosarcoma.

In addition, Yang et al. have shown that Tomentosin inhibits cell proliferation and induces apoptosis in the MOLT-4 cell line of human leukemia through inhibition of the mTOR/PI3K/Akt signalling pathway.

To the present inventors' knowledge, however, no information or description exists which demonstrates that Tomentosin is advantageously capable of acting against the lymphoid cell lines that cause the onset or development of lymphoid hematic tumors.

### SUMMARY OF THE INVENTION

In light of the above, and because of the high resistance of lymphoproliferative diseases to the drugs currently in use, the present inventors studied and evaluated the in vitro efficacy and toxicity of Tomentosin in this therapeutic area.

Such molecule had never been used before for this application, and the conducted tests made it possible to evaluate its efficacy and action mechanisms, as well as its toxicity.

Surprisingly, Tomentosin showed very little toxicity on healthy lymphoid cells and, advantageously, proved to be effective in inhibiting the proliferation of tumoral lymphoid cells, as will be further described hereinafter.

Therefore, such characteristics make Tomentosin a particularly promising molecule in view of an advantageous utilization thereof in the treatment of human lymphoproliferative diseases (hematic tumors) caused by lymphoid tumor cell lines.

The present invention concerns, therefore, Tomentosin and/or a suitable pharmaceutical composition for use in the treatment of lymphoproliferative diseases, hematic tumors, caused by lymphoid tumor cell lines, as set out in the appended claims 1 and 2.

Some preferred embodiments of the present invention are set out in the appended dependent claims.

The preferred embodiments of the present invention as set out in the following description are only provided as examples that shall not limit by any means the application scope of the present invention, which will become immediately clear to a qualified physician.

### BRIEF DESCRIPTION OF THE DRAWINGS

FIG. 1 describes the induction of cellular death of each cell line subjected to the treatment (red lines) in comparison with the reduction in the cellular proliferation of the same (blue lines).

FIG. 2 (blue lines) only shows the reduction in the cellular proliferation of each cell line subjected to the treatment.

### DETAILED DESCRIPTION OF THE INVENTION

Therefore, the present invention concerns at least the following items:
[1] The sesquiterpene lactone Tomentosin for use in the treatment of lymphoproliferative diseases, hematic tumors, myelomas, lymphomas, caused by lymphoid tumor cell lines.

Likewise, the present invention relates to:
[2] A pharmaceutical composition comprising Tomentosin as the active ingredient for use in the treatment of lymphoproliferative diseases, hematic tumors, myelomas, lymphomas, caused by lymphoid tumor cell lines, as described in [1].
[3] A composition according to [2], for use in the treatment as described in [2], wherein Tomentosin is comprised in such amounts as to allow the realization of pharmaceutical forms suitable for a total daily administration of 2-60 mg/kg of body weight of the active ingredient; preferably, 3-50 mg/kg pro die; more preferably, 4-40 mg/kg pro die; even more preferably, 5-30 mg/kg pro die.
[4] A composition according to [3], for use in the treatment as described in [2], wherein said daily administration occurs in a single administration, or is divided into plural administrations, depending on the patient's needs.
[5] A composition according to any one of items [2]-[4], for use in the treatment as described in [2], formulated in such a way that it can be administered orally and/or by injection, depending on the patient's needs.
[6] A composition according to any one of items [2]-[5], for use in the treatment as described in [2], further comprising co-formulants, excipients, additives, adjuvants, preservatives, disintegrating agents, sweeteners, flavorings and so on, according to the common practice of the pharmaceutical formulative technique of the known art.
[7] A composition according to any one of items [2]-[6], for use in the treatment as described in [2], further comprising at least another known active ingredient usable in the treatment according to [2].

The present invention is also directed to other similar therapeutic applications within the same area, as the skilled physician will be able to identify based on his/her own professional experience.

### EXPERIMENTAL SECTION

The following experimental section will synthetically illustrate, by way of example and without by any means limiting the broad application potential of the invention, some significant tests conducted in vitro which demonstrate the utility and efficacy of Tomentosin for use in the treatment of lymphoproliferative diseases, as previously described herein. Of course, a skilled physician will be able to, based on this information and his/her own experience, make use of the teaching of the present invention also for other possible applications, without however departing from the scope thereof.

The test described below made it possible to evaluate the cytotoxicity of the sesquiterpene molecules Tomentosin and Inuvisculide by using as a positive control the chemotherapy molecule Cisplatin, whose cytotoxic and chemotherapy effects are well known.

The molecules were tested at 7 different concentrations: 50 μM, 25 μM, 12.5 μM, 6.25 μM, 3.125 μM, 1.56 μM, 0.75 μM, in culture with lymphoid tumor (myeloma, lymphoma) cell lines.

Tested cell lines:
MM.1S (of multiple myeloma), cultivated in RPMI-1640 medium, 10% FBS, L-Glutamine, AA
RPMI-8226 (of multiple myeloma), cultivated in RPMI-1640 medium, 10% FBS, L-Glutamine, AA;
Raji (of Burkitt lymphoma), cultivated in RPMI-1640 medium, 10% FBS, L-Glutamine, AA;
MRC5 (of pulmonary fibroblasts), cultivated in DMEM medium, 10% FBS, L-Glutamine, AA, Pyruvate Sodium, NEAA.

The MRC5 cell line was used as a control to evaluate if these molecules were toxic also towards non-tumor cells.

### Experimental Design

The cells were seeded in 384-well plates and treated with 7 different concentrations (as indicated above) of Tomentosin, Inuviscolide and Cisplatin (positive control).

After 48 hours of treatment, drug-induced cellular death was estimated by measuring the intensity of the fluorescence signal after staining the dead cells using a known method (CellTox Green Dye—Promega).

Soon afterwards, the number of live cells was evaluated by adding CellTiter Glo P (Promega) reagent, through which the cell lysis and the luminescence signal are directly proportional to the ATP levels in each sample.

A preliminary assay was also carried out in order to select the most appropriate number of cells to be seeded in the plates for the proliferation test.

Cell Cultures

As mentioned above, commercially available MM.1S, RPMI-8226m, Raji and MRC5 cells were used.

The MM.1S, RPMI-8226 and Raji cells were cultured in RPMIl640 (Euroclone ECB9006L) culture medium supplemented with 10% FBS (Euroclone ECS0180L), 2 mM L-Glutamine (Sigma G7513) and Antibiotic Antimycotic Solution (AA, Sigma A5955).

The MRC5 cells were cultured in DMEM culture medium with high glucose content (Euroclone ECM0101L), supplemented with 10% FBS, 2 mM L-Glutamine (Sigma G7513), Non-essential aminoacids (NEAA, Sigma M7145), Pyruvate Sodium 1 mM (Sigma S8636), Antibiotic Antimycotic Solution (AA, Sigma A5955). The cells were cultivated at 37° C. in the presence of air humidified to 5% with CO2.

Cell Proliferation Validation Test)

A preliminary assay was carried out to identify the number of cells to be seeded in order to obtain a direct correlation between the luminescence measured with the CellTiter-Glo assay and the number of cultured cells.

Nine serial 1.35× dilutions of each cell line were seeded in 384-well plates; said dilution ranged from 10,000 to 0.0001000 cells per well. Four technical replicates were seeded for each condition. After 72 hours, CellTiter-Glo (Promega G7571) was added to each well, each sample was mixed 5 times, and 10 minutes later luminescence was read.

Cell Proliferation and Cytotoxicity Test

24 hours prior to the addition of the compound under examination, the cells resuspended at adequate concentrations were seeded in 384-well flat clear bottom black polystyrene TC-treated microplates (Corning 3764).

Each well contained 20 μL of cell suspension. The day after, μL of serial dilutions of the molecules under examination, with integrated CellTox (Promega G8731) green dye, were added to each sample to obtain the desired treatment concentrations.

The seeding of the cells, the preparation of the serial dilutions, and the addition of the molecules to the cells were performed using an automated liquid pipetting platform (Gilson Pipetmax).

48 hours after the treatment, the intensity of the global green fluorescence in each well was quantified using Biotek Cytation 5. For each sample, four images were taken to cover the whole area of the well. Image merging, processing and background removal were performed using the Gen5 software. Immediately after the fluorescence reading, 30 μL of CellTiter Glo reagent (Promega G7571) just prepared were added to each well, and each sample was mixed 5 times. Ten minutes later, a luminescence reading was taken. Three technical replicates for each condition under examination were plated.

Technical Notes about the Reagents

CellTiter-Glo is a reagent used for determining the number of viable cells in a culture based on the quantification of ATP, which signals the presence of metabolically active cells.

The reagent is added directly to the samples, resulting in cell lysis and generation of a luminescent signal that is proportional to the quantity of ATP. The luminescence signal is quantified using Promega Glo-Max Discover.

The CellTox green dye measures the changes that have occurred in the integrity of the membrane following cell death. The test uses a dye which is excluded from viable cells while coloring the DNA of dead cells. When the dye binds to the DNA of the compromised cells, the fluorescent properties of the dye are enhanced, whereas viable cells produce no perceptible increase in fluorescence. Therefore, the fluorescent signal produced by the dye is proportional to cytotoxicity. Fluorescence intensity (GFI) is quantified by means of a Biotek Cytation 5 wide-field automated digital microscope, led cube 465 nm, filter cube excitation 469±25 nm, emission 525±25 nm.

Cell viability is calculated as relative cell count (R):

R=T/U×100

where T is the number of cells in the treated sample and U is the number of cells in the untreated sample.

For example, a value of R=70 indicates that the number of cells at that treatment concentration is 70% of the untreated control, and therefore the number of cells has been reduced by 30%. In general, a value of R in the range of 80 to 50 indicates a slight reduction in the number of cells, a value in the range of 50 to 10 indicates a biologically significant reduction in the number of cells, and a value <10 indicates a considerable reduction in the number of cells caused by the treatment. R is used for calculating IC50 (half the maximum inhibitory concentration).

Half the maximum inhibitory concentration (IC50) is the concentration which is necessary in order to reduce the number of cells by 50%. It is calculated by entering a linear regression model into a scatter diagram wherein x=log2 drug concentration and y=relative cell number.

Cell death is measured as green fluorescence intensity (GFI).

Interpretation and Importance of R² for the Calculation of IC50

The numerical value of the R² parameter quantifies the correctness of the model. It is a value in the range of 0.0 to 1.0 with no unit of measurement. Higher values indicate that the model is more concordant with the observed data.

When R²=1.0, all observed data lie exactly on the curve with no scattering.

Induction of Cell Death

Cell death, which is directly proportional to Green Fluorescence Intensity (GFI), was computed using the method known in the art for such purpose.

Each cell line shows a basal value and a distinct GFI value (MM·1S 1.5→▮109, RPMI-8226 3.7→▮108, Raji 2.8→▮108, MRC5 5.5→▮107).

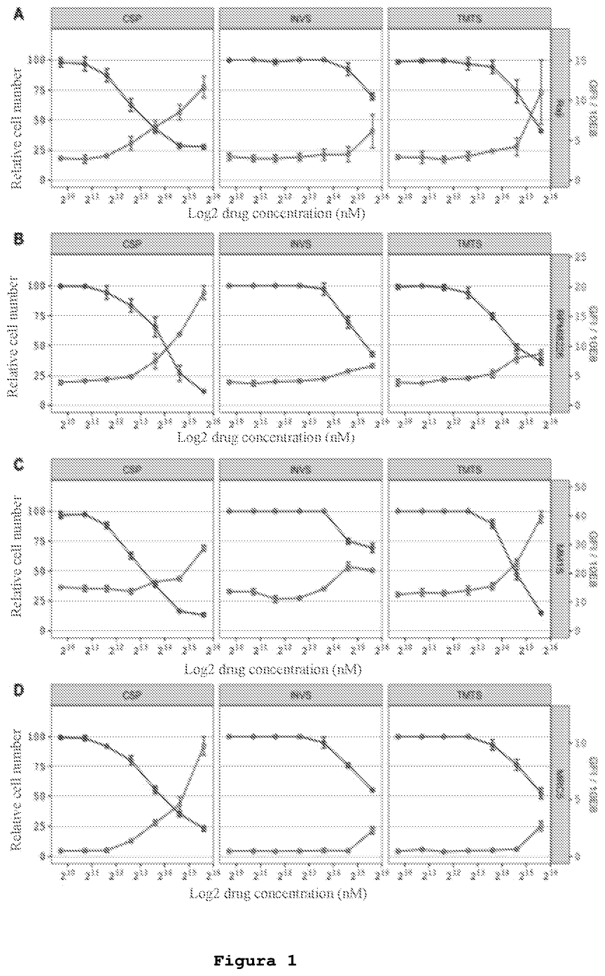

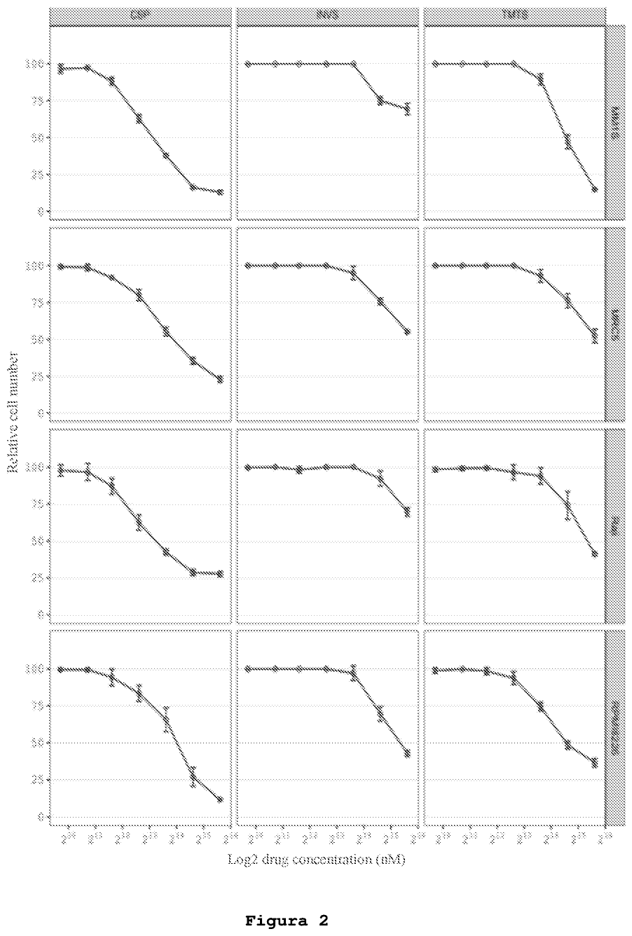

The induction of cell death in each cell line subjected to the treatment is shown as a red line in the annexed FIG. 1. The blue lines indicate the reduction in cell proliferation, as shown in the next FIG. 2.

### CONCLUSIONS

The results obtained confirmed that cysplatin (positive control) interferes with DNA replication and, as is known, kills all proliferating cells (tumor and non-tumor cells) indiscriminately. As a consequence, reduced cell proliferation and increased cell death were observed in all cell lines under examination. As a reference, the IC50 value determined for cysplatin after 72 hours of culture turned out to be 12.3 μM in the MM.1S cells, 14.9 μM in the RPMI-8226 cells, 25.7 μM in the Raji cells.

In its turn, Tomentosin showed a cytotoxic effect on the MM.1S and Raji cells, and a cytostatic effect on the RPMI-8226 cells, while it had a low anti-proliferative activity on the MRC5 non-tumor cells (with IC50>the maximum concentration).

On the contrary, Inuvisculide showed a cytostatic effect on the RPMI-8226 cells only, and low anti-proliferative activity towards the other cell lines (with IC50>the maximum concentration).

This proves that Tomentosin is active against lymphoid tumor cell lines and is not toxic for non-tumor cells, thus being an extremely promising active ingredient which could be used to advantage in the treatment of lymphoproliferative diseases.

### Industrial Applicability of the Invention

Tomentosin has proven to be significantly active in inhibiting the replication of lymphoid tumor cell lines. As a result, its pharmaceutical compositions can be used to advantage in the treatment of lymphoproliferative diseases, such as hematic tumors, e.g. myelomas and/or lymphomas, caused by lymphoid cell lines.

1. The sesquiterpene lactone Tomentosin for use in the treatment of lymphoproliferative diseases, hematic tumors, myelomas, lymphomas, caused by lymphoid tumor cell lines.

2. A pharmaceutical composition comprising Tomentosin as the active ingredient for use in the treatment of lymphoproliferative diseases, hematic tumors, myelomas, lymphomas, caused by lymphoid cell lines according to claim 1.

3. The composition according to claim 2, for use in the treatment of lymphoproliferative diseases, hematic tumors, myelomas, lymphomas, caused by lymphoid cell lines, in which Tomentosin is comprised in such amounts as to allow the realization of pharmaceutical forms suitable for a total daily administration of 2-60 mg/kg of body weight of the active ingredient.

4. The composition according to claim 3, for use in the treatment of lymphoproliferative diseases, hematic tumors, myelomas, lymphomas, caused by lymphoid cell lines, in which said daily administration occurs in a single administration, or is divided into plural administrations, depending on the patient's needs.

5. The composition according to claim 2, for use in the treatment of lymphoproliferative diseases, hematic tumors, myelomas, lymphomas, caused by lymphoid cell lines, formulated in such a way that it can be administered orally and/or by injection, depending on the patient's needs.

6. The composition according to claim 2, for use in the treatment of lymphoproliferative diseases, hematic tumors, myelomas, lymphomas, caused by lymphoid cell lines, further comprising co-formulants, excipients, additives, adjuvants, preservatives, disintegrating agents, sweeteners, flavorings and so on, according to the common practice of the pharmaceutical formulative technique of the known art.

7. The composition according to claim 2, for use in the treatment of lymphoproliferative diseases, hematic tumors, myelomas, lymphomas, caused by lymphoid cell lines, further comprising at least another known active ingredient usable in the treatment of lymphoproliferative diseases, hematic tumors, myelomas, lymphomas, caused by lymphoid cell lines.
